SEQUENCE LISTING (6 sequences)

SEQ ID NO: 1 - protein, 24 aa, Homo Sapiens
  SEQ ID NO1 - Human
IYIWAPLAGTCGVLLLSLVITLYC

SEQ ID NO: 2 - protein, 42 aa, Homo Sapiens
  SEQ ID NO2 - Human
KRGRKKLLYIFKQPFMRPVQTTQEEDGCSCRFPEEEEGGCEL

SEQ ID NO: 3 - protein, 41 aa, Homo Sapiens
  SEQ ID NO3 - Human
RSKRSRLLHSDYMNMTPRRPGPTRKHYQPYAPPRDFAAYRS

SEQ ID NO: 4 - protein, 112 aa, Artificial Sequence
  SEQ ID NO4 - Synthetic
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 170 aa, Homo Sapiens
  SEQ ID NO5 - Human
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 6 - protein, 348 aa, Artificial Sequence
  SEQ ID NO6 - Synthetic
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2019/0117691 A1
(43) Pub. Date: Apr. 25, 2019

(54) PD-1 CAR-T CELL, PREPARATION METHOD THEREFOR, AND APPLICATION THEREOF

(71) Applicant: ASCLEPIUS (Suzhou) TECHNOLOGY COMPANY GROUP CO., LTD., Suzhou City (CN)

(72) Inventor: HUASHUN LI, Suzhou (CN)

(73) Assignee: ASCLEPIUS (Suzhou) TECHNOLOGY COMPANY GROUP CO., LTD., Suzhou City (CN)

(21) Appl. No.: 16/093,322

(22) PCT Filed: Sep. 19, 2016
(86) PCT No.: PCT/CN2016/092578

§ 371 Date: Oct. 12, 2018

(30) Foreign Application Priority Data
Apr. 13, 2016 (CN) 201610226230.9

(51) Int. Cl. A61K 35/17; A61P 35/00; C12N 15/85; C12N 15/62

(52) U.S. Cl. CPC A61K 35/17; C12N 15/62; C12N 15/85; A61P 35/00

(57) ABSTRACT

Provided are a PD-1 CAR-T cell, a preparation method thereof, and an application thereof. By means of chimeric antigen receptor-modified T cell transformation, PD-1-CD8™-4-1BB-CD3ζ molecules are expressed in a T cell. The CAR-T cell prepared by the method can specifically recognize and bind with tumor cells with high PDL-1 protein expression, and is applicable to preparation of a drug for preventing and treating tumor diseases.

### FIELD OF THE INVENTION

The invention relates to the field of cellular drug for treating tumor, in particular to a PD-1 CAR-T cell and a preparation method and application thereof.

### BACKGROUND OF THE INVENTION

With the gradual progress of tumor immunotherapy research, programmed death growth factor-1 (PD-1/CD 279) and its ligand PD-L1/2 (B7-H1/CD274) won the favor of many researchers as important members in the tumor microenvironment. On Sep. 4, 2014, the U.S. Food and Drug Administration (FDA) approved Keytruda (pembrolizum ab) for the treatment of terminal or unrespectable melanoma patients who do not respond to other drugs, and Keytruda became the first drug approved by FDA to block the PD-1 cell pathway. PD-1 was first discovered in 1992, and mainly expressed in T cells, regulatory T cells, “depleted” T cells, B cells, activated mononuclear cells, dendritic cells, natural killer cells, natural killer T cells and so on. PD-1 is generally expressed in activated T cells, which includes a transmembrane region, a stem region, an Ig superfamily region, and an intracellular region comprising ITIM, ITSM. PD-1 is a synergistic inhibitory receptor, and has two ligands which are PD-L1 and PD-L2, respectively. PD-L1 is abnormally expressed in different malignant tumors, such as squamous cell carcinoma in lung, esophagus, head and neck, and in other types of malignant tumors, for example, ovarian cancer, bladder cancer, malignant melanoma, and glioma. Structurally, PD-L2 is similar to PD-L1, both being type I transmembrane proteins which include a signal peptide, an IgV-like region, an IgC-like region, a stem region, a transmembrane region, and a cytoplasmic region. PD-1 binds to the ligand PD-L1/2 to phosphorylate the tyrosine in ITIM and ITSM, and promotes the binding of SH P-1 and SH P-2 to ITIM and IT SM, which in turn delivers T cell inhibitory signals and indirectly leads to cell death by down-regulating expression of BC L·X L and differentiation of T cells. The PD-1 PD-L1/2 pathway is also thought to be a pathway that mediates immunosuppression, with PD-1 working as a negative regulatory checkpoint. The inhibitory function of PD-1 and PD-L1 pathways can enhance T cell responses in vitro; PD-1 works by binding to specific ligands (PD-L1, PD-L2) in vivo, which down-regulates antigen-stimulated lymphocyte proliferation and the production of cytokines, and ultimately leads to the “depletion” of lymphocytes and the induction of immune tolerance. Tumor cells in solid tumors can up-regulate the expression of PD-L1, and then provide an inhibitory signal which down-regulates the activated T cells, ultimately turning off immune response and inducing immune tolerance. The survival rate of patients with high PD-L1 expression level was significantly decreased, which was associated to and consistent with most reports describing the association of high expression level of PD-L1 on most tumor with poor prognosis. In addition to expression in malignant melanoma, PD-L1 also was expressed in other different tumors, including glioblastoma, pancreatic cancer, ovarian cancer, breast cancer, renal cell carcinoma, head and neck and esophageal squamous cell carcinoma, and non-small cell lung cancer, and high expression of PD-L1 on tumor cells is associated with poor prognosis.

The theory of chimeric antigen receptor T lymphocyte technology (CAR-T) is that T cells modified by a chimeric antigen receptor can specifically recognize tumor-associated antigens, which makes targeting ability, killing activity and persistence of effector T cells higher than that of the conventionally used immune cells, and can overcome the local immunosuppressive microenvironment of the tumor and break the host immune tolerance state. Normally, the body's T lymphocytes recognize the target cells through the T cell receptors on their surface. This recognition is specific. That is, a T lymphocyte only recognizes target cells with specific antigens, and these specific antigens are presented to T lymphocytes under the action of special molecules after being processed in cells. These antigen-presenting molecules are either present on the surface of antigen presenting-cells or on the surface of target cells, which means that the activation of the T cell requires not only specific recognition antigen but also costimulatory signals. In tumors: (1) Tumor cells must be presented by antigens before they are recognized by T cell receptors (specific signals); (2) There's a second signal involved (As shown in the diagram, CD28 participates), and CD28 must also be activated. After the first and second signals are both activated, T cells can only kill the tumor.

However, tumors realize immune escape mainly from two aspects: (1) The antigen presentation mechanism of tumor cell is down-regulated or even lost (HLA-negative), which causes T cells to fail to recognize tumor cells; (2) Many tumor cells are abnormally highly expressed PD-L1 molecules, which activates PD-1 molecules on the surface of T cells, and leads to the depletion of T cells function and even death of T cells. Based on this situation, scientists have proposed the concept of constructing a chimeric T cell receptor (now commonly referred to as a Chimeric Antigen Receptor). Chimeric Antigen Receptor (CAR) is mainly composed of two parts, one end of which is located outside the cell that can specifically recognize an antigen on the surface of cancer cells, the other end of which is located in the cell that contains a signal activation element (such as a T cell receptor, Zeta chain), which plays a role in transmitting signals and activating T cells. Therefore, the T-lymphocytes (CAR-T cells) expressing CAR can prevent the T cell receptor from recognizing the restriction of target cells, and play a killing role in targeting cancer cells.

Construction of CAR-T can make T cells recognize and kill cancer cells without restriction, and bring the inherent killing function of T cells into full play. At present, researchers have designed CAR-T cells for various tumor-associated antigens, such as CD138, CD19, ErbB2, EGFRvIII, cell-surface glycoprotein (CS1), GD2, CD20, etc. However, most of these CAR-T cells are still in the research stage, and their the clinical effects need to be confirmed. Therefore, it is necessary to design and construct novel CAR-T cells to achieve breakthroughs in the treatment of solid tumors.

### SUMMARY OF THE INVENTION

The main technical problem to be solved by the present invention is to provide a PD-1 CAR-T cell and preparation method and application thereof, wherein the obtained PD-1 CAR-T cells can specifically recognize and bind to tumor cells with high expression of PDL-1 protein and can be used in the preparation of drugs for the prevention and treatment of tumor diseases.

In order to solve the above technical problem, one technical solution adopted by the present invention is to provide a PD-1 CAR-T cell, wherein a PD-1-CD8™-4-1BB-CD3ζ fusion protein is expressed in the PD-1-CAR-T cell.

In a preferred embodiment of the present invention, the PD-1 CAR-T cell is manufactured by:

(1) synthesizing and amplifying the gene encoding the PD-1-CD8™-4-1BB-CD3ζ fusion protein and cloning the gene encoding the PD-1-CD8™-4-1BB-CD3ζ fusion protein into a lentiviral expression vector;

(2) using a lentivirus envelop plasmid and the lentiviral expression vector of step (1) to infect a 293T cell, packaging and preparing a lentivirus;

(3) isolating and expanding human peripheral blood T lymphocytes, and infecting the T lymphocytes with the lentivirus of step (2) to obtain the PD-1 CAR-T cell expressing the PD-1-CD8™-4-1BB-CD3ζ fusion protein.

In a preferred embodiment of the invention, said PD-1 is expressed on a surface of the said PD-1 CAR-T cell, and said 4-1BB-CD3ζ molecule transmits the T cell activating signal inside said PD-1 CAR-T cell.

In a preferred embodiment of the present invention, the amino acid sequence of PD-1 in the PD-1-CD8™-4-1BB-CD3ζ fusion protein is SEQ ID NO:5; and the amino acid sequence of CD8™ in the PD-1-CD8™-4-1BB-CD3ζ fusion protein is SEQ ID NO: 1.

In a preferred embodiment of the present invention, the amino acid sequence of 4-1BB in the PD-1-CD8™-4-1BB-CD3ζ fusion protein is SEQ ID NO:2; wherein the 4-1BB in the PD-1-CD8™-4-1BB-CD3ζ fusion protein can be replaced by CD28 that has the amino acid sequence of SEQ ID NO:3.

In a preferred embodiment of the present invention, the amino acid sequence of the CD3ζ in the PD-1-CD8™-4-1BB-CD3ζ fusion protein is SEQ ID NO: 4; wherein the T cell is derived from human periphery blood T lymphocytes.

In a preferred embodiment of the invention, the amino acid sequence of the PD-1-CD8™-4-1BB-CD3ζ fusion protein is SEQ ID NO:6.

In a preferred embodiment of the invention, the PD-1 CAR-T cell is used in the preparation of an anti-tumor drug.

In a preferred embodiment of the invention, the PD-1 CAR-T cell is used in the preparation of therapeutic drugs that target tumors with high expression of PD-L1.

The beneficial effects of the present invention are: the PD-1 CAR-T cells of the present invention and preparation methods and applications thereof, including modifying and altering T cells with chimeric antigen receptors and expressing a PD-1-CD8™-4-1BB-CD3ζ molecule in the T cells, so that the modified T cells can specifically recognize and kill tumors, and the obtained cells have more efficient tumor killing activity.

### BRIEF DESCRIPTION OF THE DRAWINGS

In order to more clearly illustrate the technical solutions in the embodiments of the present invention, the drawings used in the description of the embodiments will be briefly described below. It is obvious that the figures in the following description are only some embodiments of the present invention. For a person of ordinary skilled in the art, other drawings can be obtained based on these figures without any creative work. The figures include:

FIG. 1 illustrates a map of a PRRLSIN-PD-1 lentiviral plasmid vector;

FIG. 2 illustrates a flow cytometry result of MCF7-PDL1 engineering cell line with high expression level of PD-L1;

FIG. 3 illustrates a flow cytometry result of the proportion of T cells two days after PBMC activation;

FIG. 4 illustrates a viral infection effect diagram after PD-1-CART was infected with different volumes of virus for 14 days;

FIG. 5 illustrates a result of the CAR-PD1 killing experiment in vitro: a diagram of different ratios of effect;

FIG. 6 illustrates a diagram of PD-1-CAR-T cell proliferation;

FIG. 7 illustrates a diagram of PD-1-CAR-T cells killing effect in vitro under different effect-target ratio conditions.

### DETAILED DESCRIPTION OF THE INVENTION

The technical solutions in the embodiments of the present invention are clearly and completely described below. It is obvious that the described embodiments are only a part of the embodiments of the present invention, not all of the embodiments. Based on the embodiments of the present invention, all other embodiments obtained by a person skilled in the art without any creative work is within the scope of the protection of the present invention.

### Embodiment 1

### Preparation of a Lentiviral Expression Vector

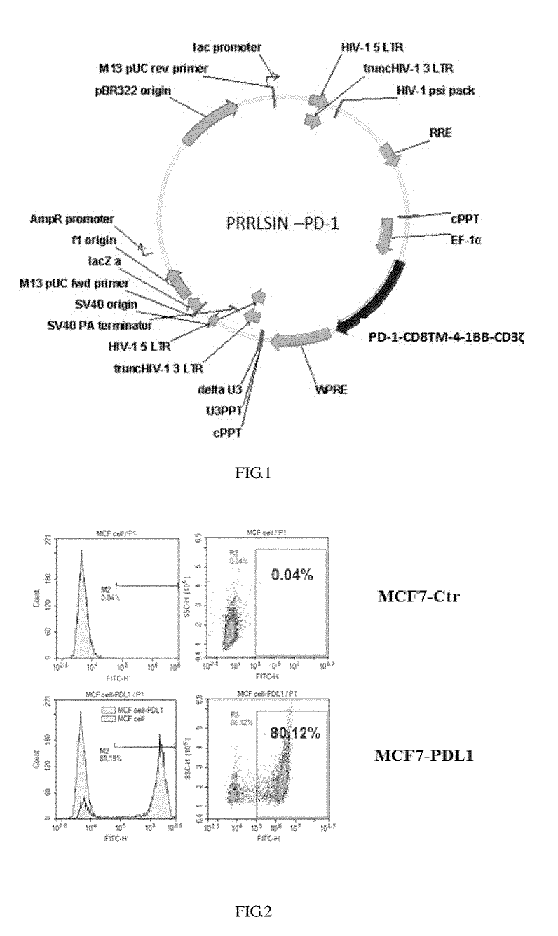

The gene encoding the PD1-CD8™-4-1BB-CD3ζ was synthesized, then the gene was ligated into the PRRSLIN vector by enzyme restriction and transformation, and the upstream of the gene is EP-1α promoter. The vector was transformed into Stbl3 Escherichia coli strain, and screened by ampicillin to obtain positive clones, then the plasmids were extracted and identified by restriction enzyme digestion, and PRRLSIN-PD-1 lentiviral transfection vector was obtained, the structure of which is as shown in FIG. 1.

### Embodiment 2

### Preparation of Lentivirus

(1) Twenty-four hours before transfection, seeding 293T cells into 15 cm culture dishes at a cell density of approximately 8×10⁶ cell per dish, which could ensure that the cells are at about 80% of confluence and distributed uniformly in the culture dish during transfection.
(2) Prepare solution A and solution B
Solution A: 6.25 ml of 2×HEPES buffer (using 5 large dishes that are packed together could achieve the best effects).
Solution B: adding the following plasmids, respectively, and mixing: 112.5 μg of pRRLSIN-EF-ROBO1 (target plasmid); 39.5 μg of pMD2.G (VSV-G envelop); 73 μg of pCMVR8.74 (gag, pol, tat, rev); 625 μl of 2M calcium ion solution. Total volume of solution A: 6.25 ml.

The solution B was mixed completely, and the solution A was added dropwise while the solution A was gently rocked, then let the solution sit for 5-15 minutes. The above mixed solution of A and B was gently rocked and added to the dish containing 293T cells dropwise, then the culture dish was gently shaken back and forth to distribute the mixture of DNA and calcium ions evenly. The culture dish was placed in an incubator to incubate for 16-18 hours (do not rotate the culture dish). Fresh medium was replaced and continued incubating, then the supernatant containing virus was collected after 48 hours and 72 hours, respectively. The supernatant containing virus was observed by fluorescence microscopy, more than 95% of the cells should show green fluorescence. The supernatant was centrifuged at 500 g for 10 minutes at 10° C., followed by being filtered with PES membrane (0.45 μm). Beckman Coulter Ultra-clear SW28 centrifuge tubes were sterilized with 70% ethanol, and sterilized under UV light for 30 minutes. The filtered supernatant containing lentivirus was transferred to a centrifuge tube. A layer of 20% sucrose was carefully spread on the bottom of the centrifuge tube (1 ml of sucrose was added per 8 ml of supernatant). The centrifuge tube was equilibrated with PBS, and centrifuged at 25,000 rpm (82, 700 g) for 2 hours at 4° C. The centrifuge tube was taken out carefully, and the supernatant was poured off, followed by being inverted to remove residual liquid. 100 μl of PBS was added in the centrifuge tube and sealed, then placed at 4° C. for 2 hours, gently rocked once per 20 minutes during the time, followed by being centrifuged for 1 minute (25° C.) at 500 g, and the virus supernatant was collected. After being cooled on ice, the virus supernatant was stored at −80° C.

### Embodiment 3

### Preparation of PD-1 CAR-T Cells

0.5 ml of blood was taken and tested for pathogenic microorganisms rapidly to exclude microbial infections such as HBV, HCV, HDV and HEV, HIV-1/2, treponema pallidum and parasites; 50 ml of blood was collected with heparin bottle (heparin anticoagulation) under sterile conditions, and immediately (4° C., within 24 hours) sent to the cell preparation laboratory to ensure that this process was free of pathogenic microbial contamination. After the patient's blood was obtained, the surface of the heparin bottle was wiped with an alcohol cotton ball for disinfection in the GMP preparation room, then the heparin bottle was placed in a biological safety cabinet. Two 50 ml centrifuge tubes were opened in advance, then the blood was transferred into the two 50 ml centrifuge tubes and tightened up. The above 50 ml centrifuge tubes filled with blood were placed in a centrifuge and centrifuged at 400 g (2000 rpm) for 10 min at room temperature, then the supernatant plasma was collected and the precipitate layer was removed after centrifugation. The collected autologous plasma was inactivated at 56° C. for 30 minutes. After being stood for 15 minutes at 4° C., the collected autologous plasma was centrifuged at 900 g for 30 min at 4° C. to take the supernatant for use. The enriched blood cells above were diluted to 30 ml/tube with physiological saline, and two new 50 ml centrifuge tubes were opened, then 15 ml of human lymphocyte separation liquid was added to each centrifuge tube. The diluted blood cell solution was slowly added to the centrifuge tube which contains the human lymphatic separation solution with a pipette, and tightened up. It is noted that the blood should be added to the upper layer of the lymphatic separation solution, and the interface of the human lymphatic separation solution should not be broken. The added blood cell solution was placed in a centrifuge which was adjusted to a minimum rate of rise-and-fall, then the added blood cell solution was centrifuged at 400 g (2000 rpm) for 20 min at room temperature. The middle white blood cell layer of two tubes was collected in a 15 ml sterile centrifuge tube, and 5 ml of physiological saline was added, and then washed twice (the collected middle white blood cell layer was centrifuged at 400 g for 10 minutes) to obtain peripheral blood mononuclear cells (PBMC). Complete growth medium was made, the concentration of V-VIVO15 added autologous AB (FBS) was 5%, the concentration of IL-2 was 40 ng/ml, and the isolated PBMC was diluted to 2×10⁶/ml with medium, then 50 μl was taken, and the T cells purity of PBMC was detected by flow cytometer. On 0 day, Buffer1 was made that 1% FBS was added to PBS, the beads were rocked for 30 s or manually shaken up and down for 5 min CD3/CD28 beads were taken out according to the ratio of beads to T cells of 3-1, and the beads were put in 1.5 ml EP tube, followed by adding 1 ml buffer 1 to clean the beads. After that, the beads were suck from the EP tube for 1 min with magnet and washing solution was discarded, which was repeated twice, then the beads were re-suspended to the original volume with the medium, and the cells and beads were mixed, followed by being added in a suitable culture bottle in 2×10⁶ PBMC/ML. On the second day, the density of cells was adjusted to 3-5×10⁶/ml, and the virus vector was added in the proportion of virus vector:cell of 1:5, meanwhile, 4 μg/ml and 40 ng/ml IL⁻² of polybrene were added. Fresh complete medium was added after 4 hours, and the density of cells was adjusted to 1×10⁶/ml to continuous culture. All the cells were centrifuged, and fresh medium was added to continuous culture. Half a volume change was replaced per 2-3 days to maintain the density of the cell in 0.5-1×10⁶/ml. When the number of cells reached 10⁹ in the period of 10-12 days, the cells were centrifuged at 400 g for 5 min to get immune cells, followed by being washed twice with pre-cooled PBS (400 g, 5 min). The cells were count by hemocytometer, and the cell group and the proportion of CART cells were detected by flow cytometer. The color change, cell density, and cell morphology of the medium was observed daily and recorded accordingly. The interleukin 2 which is required by the total volume was added in the process of gradually expanding cultivation.

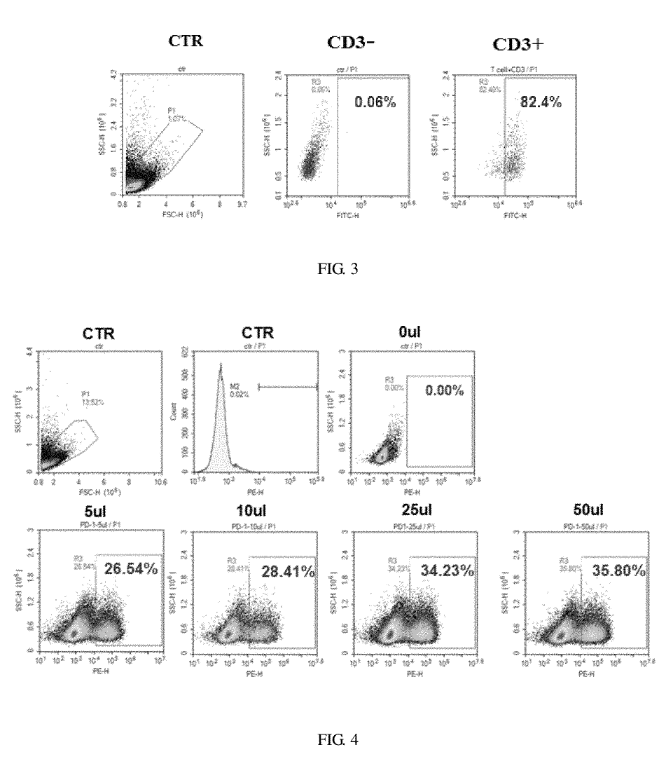

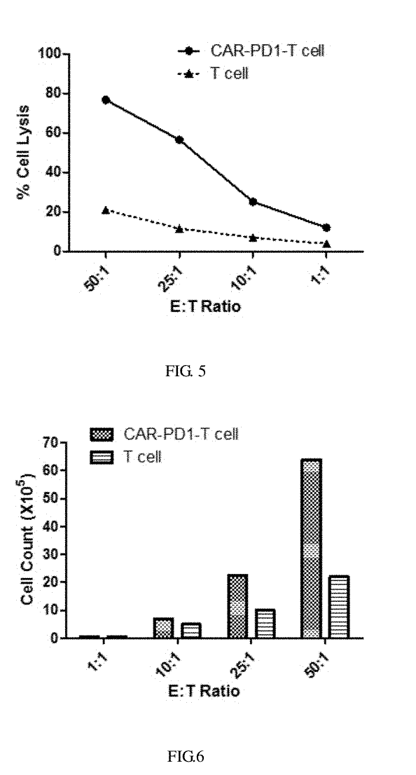

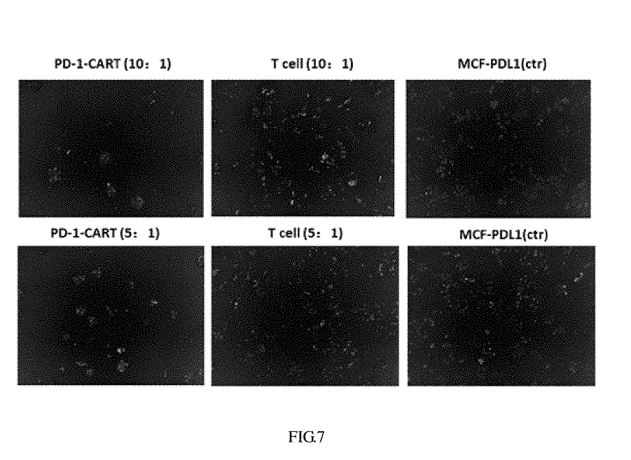

The results showed that the proportion of T cells detected by flow cytometry reached more than 80% (see FIG. 3) after two days of PBMC activation. After virus infection of T cells, the infection effects of PD-1-CART with different volumes virus at 14-day were detected. The results showed that 35.8% of the cells were successfully infected (FIG. 4) and PD-1-CART cells were prepared.

### Embodiment 4

### Construction and Detection of Engineering Cell Lines

(1) Preparation of lentivirus PD-L1 (The specific preparing method is as the method in the second embodiment);
(2) Infection of MCF cells: 500,000 MCF7 cells were inoculated in 6-well plates the day before infection. When the cells grow to 80% on the second day, 500 μl of packaged PD-L1 virus was added in a 6-well plate, meanwhile, control cell (no virus added) was set, culture medium was changed after 12-16 hours, and then the positive cells of PD-L1 were sorted by flow cytometer 3 days after infection;
(3) Detection of engineered cell lines: 20,000 cells were taken from the sorted positive cells of PD-L1, followed by being centrifuged at 400 g for 5 min, then washed twice with pre-cooled PBS, and 2.5 ul of PD-L1 antibody (Biolegend) was added and incubated in the dark for 20 min, after that, centrifuged and washed once with pre-cooled PBS, then the cells was re-suspended with 100 μl PBS, and the expression of PD-L1 was detected by flow cytometer, see FIG. 2. The experimental results confirmed that the engineered cell lines were successfully constructed, which can be used as a target cell for subsequent killing experiments.

### Embodiment 5

### Activity Assay of PD-1 CAR-T Cells In Vitro

ELISA was used to detect LDH release, which was to detect the killing effect of PD-1 CAR-T cells on MCF7-PD-L1 target cells.

(1) Adjusting the target cells to 5×10⁴/ml with RPMI-1640 medium containing 5% calf serum.
(2) Adding target cells to 96-well cell culture plates, and adding 100 μl to each well. Three effector cells naturally released control wells were only added 100 μl of culture solution without adding target cells.
(3) Adding 100 μl of effector cells to each well, and the ratio of effector cells to target cells was 50:1; 25:1; 10:1; 5:1; or 1:1. Natural release wells were only added 100 μl of culture medium without effector cells, and incubating the effector cells and the target cells for 6 hours. Meanwhile, setting up three replicate wells for each experiment.
(4) Adding 10 μl Lysis Solution (10×) to the largest release well (positive control), and incubating for 45 min-60 min Meanwhile, placing three replicate wells each experiment.
(5) Taking out 50 μl of the test sample and the control sample in the above 3 and 4 steps, respectively, and adding in the fresh 96-well microtiter plate, then adding the assay buffer and the substance mix, followed by being protected from light for 30 minutes.
(6) Adding 50 μl stop solution.
(7) Measuring absorbance value at 490 nm or 492 nm in an hour.
(8) Killing rate=experimental group LDH (OD)/Max LDH release group (OD).
(9) Calculation formula: Killing efficiency=(experimental−effector spontaneous−target spontaneous)/(target maximum−target spontaneous)×100%.

The results showed that the prepared PD-1-CAR-T cells could significantly kill the target cell lines with high expression of PDL1, and the different proportions of PD-1 CAR-T and target cells (MCF7-PDL1) were co-incubated for 4 hours, followed by being detected by ELISA experiment. The result of ELISA experiment showed that the cell killing efficiency also increased (see FIG. 5), and microscopic imaging showed that tumor cells were obviously dead (FIG. 7) with the increasing of the E:T ratio. At the same time, different proportions of CAR-T cells were incubated with target cells (MCF7-PDL1) for 16 hours under the stimulation of the target cell strain, and the number of T cells was counted by MTT. The results showed that CAR-PD1 cells proliferated significantly with the increase of E:T ratio.

PD-1 CAR-T cells and their preparation methods can also be applied to the preparation of PD-1 CAR-NK cells, except that T cells are replaced with NK cells, and other molecular elements are unchanged.

Sequence list:

The amino acid sequence of CD8 ™ SEQ ID NO: 1 is:
IYIWAPLAGTCGVLLLSLVITLYC.
The sequence of 4-1BB SEQ ID NO: 2 is:
KRGRKKLLYIFKQPFMRPVQTTQEEDGCSCRFPEEEEGGCEL.
The sequence of CD28 SEQ ID NO: 3 is:
RSKRSRLLHSDYMNMTPRRPGPTRKHYQPYAPPRDFAAYRS.
The molecular sequence of CD3ζ SEQ ID NO: 4 is:
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR.
The sequence of PD-1 SEQ ID NO: 5 is:
MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLV.
The amino acid sequence of PD-1-CD8 ™-4-1BB-CD3ζ
fusion protein SEQ ID NO: 6 is:
MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLVIYIWAPLAGTCGVLLLSLVITLYCKRGRKK
LLYIFKQPFMRPVQTTQEEDGCSCRFPEEEEGGCELRVKFSRSADAPAYK
QGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR.

1-9. (canceled)

10. A PD-1 CAR-T cell expressing a PD-1-CD8-4-1 BB-CD3ζ fusion protein, wherein the PD-1 portion of the fusion protein is expressed on a surface of the CAR-T cell and the 4-1 BB-CD3ζ portion of the fusion protein is expressed inside the CAR-T cell.

11. The PD-1 CAR-T cell of claim 10, wherein the amino acid sequence of PD-1 in the PD-1-CD8-4-1 BB-CD3ζ fusion protein is SEQ ID NO:5; and the amino acid sequence of CD8 in the PD-1-CD8-4-1 BB-CD3ζ fusion protein is SEQ ID NO:1.

12. The PD-1 CAR-T cell of claim 10, wherein the amino acid sequence of 4-1BB in the PD-1-CD8-4-1 BB-CD3ζ fusion protein is SEQ ID NO:2; wherein the 4-1BB in the PD-1-CD8-4-1 BB-CD3ζ fusion protein can be replaced by CD28 that has the amino acid sequence of SEQ ID NO:3.

13. The PD-1 CAR-T cell of claim 10, wherein the amino acid sequence of CD3ζ in the PD-1-CD8-4-1 BB-CD3ζ fusion protein is SEQ ID NO:4.

14. The PD-1 CAR-T cell of claim 10, wherein the amino acid sequence of the PD-1-CD8-4-1 BB-CD3ζ fusion protein is SEQ ID NO:6.

15. The PD-1 CAR-T cell of claim 10, wherein the T cell is derived from human periphery blood T lymphocytes.

16. A method of making a PD-1 CAR-T cell, comprising:
(1) cloning a gene encoding a PD-1-CD8-4-1 BB-CD3ζ fusion protein into a lentiviral expression vector;
(2) packaging and preparing a lentivirus by expressing lentiviral envelop plasmid and the lentiviral expression vector of step (1) in a 293 T cell;
(3) isolating and expanding human peripheral blood T lymphocytes, and infecting the T lymphocytes with the lentivirus of step (2) to obtain the PD-1 CAR-T cells expressing the PD-1-CD8-4-1 BB-CD3ζ fusion protein; wherein the PD-1 portion of the fusion protein is expressed on a surface of the CAR-T cell and the 4-1 BB-CD3ζ portion of the fusion protein is expressed inside the CAR-T cell.

17. The method of claim 16, wherein the amino acid sequence of PD-1 in the PD-1-CD8-4-1 BB-CD3ζ fusion protein is SEQ ID NO:5; and the amino acid sequence of CD8 in the PD-1-CD8-4-1 BB-CD3ζ fusion protein is SEQ ID NO:1.

18. The method of claim 16, wherein the amino acid sequence of 4-1 BB in the PD-1-CD8-4-1 BB-CD3ζ fusion protein is SEQ ID NO:2; wherein the 4-1 BB in the PD-1-CD8-4-1BB-CD3ζ fusion protein can be replaced by CD28 that has the amino acid sequence of SEQ ID NO:3.

19. The method of claim 16, wherein the amino acid sequence of CD3ζ in the PD-1-CD8-4-1 BB-CD3ζ fusion protein is SEQ ID NO:4.

20. The method of claim 16, wherein the amino acid sequence of the PD-1-CD8-4-1 BB-CD3ζ fusion protein is SEQ ID NO:6.

21. The method of claim 16, wherein the T cell is derived from human periphery blood T lymphocytes.

22. A method of treating tumor by administering to a patient a PD-1 CAR-T cell expressing a PD-1-CD8-4-1 BB-CD3ζ fusion protein; wherein the PD-1 portion of the fusion protein is expressed on a surface of the CAR-T cell and the 4-1 BB-CD3ζ portion of the fusion protein is expressed inside the CAR-T cell.

23. The method of claim 22, wherein the amino acid sequence of PD-1 in the PD-1-CD8-4-1 BB-CD3ζ fusion protein is SEQ ID NO:5; and the amino acid sequence of CD8 in the PD-1-CD8-4-1 BB-CD3ζ fusion protein is SEQ ID NO:1.

24. The method of claim 22, wherein the amino acid sequence of 4-1 BB in the PD-1-CD8-4-1 BB-CD3ζ fusion protein is SEQ ID NO:2; wherein the 4-1 BB in the PD-1-CD8-4-1BB-CD3ζ fusion protein can be replaced by CD28 that has the amino acid sequence of SEQ ID NO:3.

25. The method of claim 22, wherein the amino acid sequence of CD3ζ in the PD-1-CD8-4-1 BB-CD3ζ fusion protein is SEQ ID NO:4.

26. The method of claim 22, wherein the amino acid sequence of the PD-1-CD8-4-1 BB-CD3ζ fusion protein is SEQ ID NO:6.

27. The method of claim 22, wherein the tumor is characterized with high expression level of PDL-1.
